SEQUENCE LISTING (29 sequences)

SEQ ID NO: 1 - DNA, 20 bp, Artificial Sequence
  forward primer for amplification specific fragments of IS6110 gene in Mycobacterium tuberculosis complex
cgaactcaaggagcacatca

SEQ ID NO: 2 - DNA, 19 bp, Artificial Sequence
  reverse primer for amplification specific fragments of IS6110 gene in Mycobacterium tuberculosis complex
gtcgaggaccatggaggtg

SEQ ID NO: 3 - DNA, 19 bp, Artificial Sequence
  forward primer for amplification specific fragments of 16S rRNA gene in Nontuberculous mycobacteria
gtggcgaacgggtgagtaa

SEQ ID NO: 4 - DNA, 18 bp, Artificial Sequence
  reverse primer(NTM-1) for amplification specific fragments of 16S rRNA gene in Nontuberculous mycobacteria
cccacaccgcaaaagctt

SEQ ID NO: 5 - DNA, 17 bp, Artificial Sequence
  reverse primer(NTM-1) for amplification specific fragments of 16S rRNA gene in Nontuberculous mycobacteria
cccacaccgcaaaagct

SEQ ID NO: 6 - DNA, 18 bp, Artificial Sequence
  reverse primer(NTM-1) for amplification specific fragments of 16S rRNA gene in Nontuberculous mycobacteria
tcccacaccgcaaaagct

SEQ ID NO: 7 - DNA, 18 bp, Artificial Sequence
  reverse primer(NTM-2) for amplification specific fragments of 16S rRNA gene in Nontuberculous mycobacteria
catcccacaccgctaccw

SEQ ID NO: 8 - DNA, 18 bp, Artificial Sequence
  reverse primer(NTM-2) for amplification specific fragments of 16S rRNA gene in Nontuberculous mycobacteria
catcccacaccgctacct

SEQ ID NO: 9 - DNA, 18 bp, Artificial Sequence
  reverse primer(NTM-2) for amplification specific fragments of 16S rRNA gene in Nontuberculous mycobacteria
catcccacaccgctacca

SEQ ID NO: 10 - DNA, 19 bp, Artificial Sequence
  forward primer for amplification specific fragments of 16S rRNA gene in Mycobacterium tuberculosis complex
gtcttgtggtggaaagcgc

SEQ ID NO: 11 - DNA, 19 bp, Artificial Sequence
  reverse primer for amplification specific fragments of 16S rRNA gene in Mycobacterium tuberculosis complex
agagaacccggaccttcgt

SEQ ID NO: 12 - DNA, 22 bp, Artificial Sequence
  forward primer for amplification specific fragments of 16S rRNA gene in Nontuberculous mycobacteria
catgtyttstggkgsaaagctt

SEQ ID NO: 13 - DNA, 19 bp, Artificial Sequence
  reverse primer for amplification specific fragments of 16S rRNA gene in Nontuberculous mycobacteria
cgtaggagtctgggccgta

SEQ ID NO: 14 - DNA, 22 bp, Artificial Sequence
  forward primer for amplification specific fragments of 16S rRNA gene in Nontuberculous mycobacteria
catgtcttgtgggggaaagctt

SEQ ID NO: 15 - DNA, 22 bp, Artificial Sequence
  forward primer for amplification specific fragments of 16S rRNA gene in Nontuberculous mycobacteria
catgttttgtgggggaaagctt

SEQ ID NO: 16 - DNA, 22 bp, Artificial Sequence
  forward primer for amplification specific fragments of 16S rRNA gene in Nontuberculous mycobacteria
catgtcttctgggggaaagctt

SEQ ID NO: 17 - DNA, 22 bp, Artificial Sequence
  forward primer for amplification specific fragments of 16S rRNA gene in Nontuberculous mycobacteria
catgtcttgtggtggaaagctt

SEQ ID NO: 18 - DNA, 22 bp, Artificial Sequence
  forward primer for amplification specific fragments of 16S rRNA gene in Nontuberculous mycobacteria
catgtcttgtggggcaaagctt

SEQ ID NO: 19 - DNA, 22 bp, Artificial Sequence
  forward primer for amplification specific fragments of 16S rRNA gene in Nontuberculous mycobacteria
catgttttctgggggaaagctt

SEQ ID NO: 20 - DNA, 22 bp, Artificial Sequence
  forward primer for amplification specific fragments of 16S rRNA gene in Nontuberculous mycobacteria
catgtcttctggtggaaagctt

SEQ ID NO: 21 - DNA, 22 bp, Artificial Sequence
  forward primer for amplification specific fragments of 16S rRNA gene in Nontuberculous mycobacteria
catgtcttgtggtgcaaagctt

SEQ ID NO: 22 - DNA, 22 bp, Artificial Sequence
  forward primer for amplification specific fragments of 16S rRNA gene in Nontuberculous mycobacteria
catgttttgtggggcaaagctt

SEQ ID NO: 23 - DNA, 22 bp, Artificial Sequence
  forward primer for amplification specific fragments of 16S rRNA gene in Nontuberculous mycobacteria
catgtcttctggggcaaagctt

SEQ ID NO: 24 - DNA, 22 bp, Artificial Sequence
  forward primer for amplification specific fragments of 16S rRNA gene in Nontuberculous mycobacteria
catgttttgtggtggaaagctt

SEQ ID NO: 25 - DNA, 22 bp, Artificial Sequence
  forward primer for amplification specific fragments of 16S rRNA gene in Nontuberculous mycobacteria
catgttttctggtggaaagctt

SEQ ID NO: 26 - DNA, 22 bp, Artificial Sequence
  forward primer for amplification specific fragments of 16S rRNA gene in Nontuberculous mycobacteria
catgttttctggggcaaagctt

SEQ ID NO: 27 - DNA, 22 bp, Artificial Sequence
  forward primer for amplification specific fragments of 16S rRNA gene in Nontuberculous mycobacteria
catgttttgtggtgcaaagctt

SEQ ID NO: 28 - DNA, 22 bp, Artificial Sequence
  forward primer for amplification specific fragments of 16S rRNA gene in Nontuberculous mycobacteria
catgtcttctggtgcaaagctt

SEQ ID NO: 29 - DNA, 22 bp, Artificial Sequence
  forward primer for amplification specific fragments of 16S rRNA gene in Nontuberculous mycobacteria
catgttttctggtgcaaagctt

(19) United States
(12) Patent Application Publication
(10) Pub. No.: US 2013/0177914 A1
(43) Pub. Date: Jul. 11, 2013

(54) METHOD FOR DETECTING MYCOBACTERIUM TUBERCULOSIS AND NONTUBERCULOUS MYCOBACTERIA USING DUPLEX POLYMERASE CHAIN REACTION

(71) Applicant: Jeong-Uk Kim, Gangneung-si (KR)

(72) Inventor: Jeong-Uk Kim, Gangneung-si (KR)

(73) Assignee: UNIVERSITY OF ULSAN FOUNDATION FOR INDUSTRY COOPERATION, Ulsan (KR)

(21) Appl. No.: 13/700,155

(22) PCT Filed: May 26, 2011
(86) PCT No.: PCT/KR2011/003857

§ 371 Date: Mar. 29, 2013

(30) Foreign Application Priority Data
May 27, 2010 (KR) 10-2010-0049690
Jun. 30, 2010 (KR) 10-2010-0062845

(51) Int. Cl. C12Q 1/68

(52) U.S. Cl. CPC C12Q 1/689
USPC 435/6.12

(57) ABSTRACT

Disclosed are primer sets specific for the IS6110 or 16S rRNA gene characteristic of MTC and for the 16S rRNA gene characteristic of NTM, kits for the detection of MTC and NTM, comprising the same, and methods for detecting MTC and NTM by duplex PCR using the same. Because the primers are exclusive to Mycobacterium tuberculosis and nontuberculous mycobacteria, the methods, which can employ an inexpensive typical PCR cycler, can be a clinical diagnostic means useful for the detection of both Mycobacterium tuberculosis and nontuberculous mycobacteria at the same time, with higher efficiency.

### TECHNICAL FIELD

The present invention relates to the detection of Mycobacterium tuberculosis and nontuberculous mycobacteria. More particularly, the present invention relates to a primer set capable of detecting specific nucleotide sequences of Mycobacterium tuberculosis and nontuberculous mycobacteria, a kit for the detection of Mycobacterium tuberculosis and nontuberculous mycobacteria, comprising the same, and a method for detecting Mycobacterium tuberculosis and nontuberculous mycobacteria simultaneously, using the same.

### BACKGROUND ART

Nontuberculous mycobacteria are widely distributed in the environment, particularly in wet soil, marshland and rivers, and had been recognized as non-pathogenic bacteria before the discovery of its opportunistic characteristics. In the 1980s, nontuberculous mycobacteria were found to be an opportunistic pathogen of pulmonary diseases in patients with acquired immunodeficiency syndrome (AIDS). Further, the bacteria were also known to cause diseases in other patients. With the report of the pathogenesis of nontuberculous mycobacteria, its clinical significance has been increasingly recognized.

Recently, the United States of America and many European countries, which have a low prevalence of tuberculosis, have seen an increase in the incidence of infections caused by nontuberculous mycobacteria. In Korea, the isolation of nontuberculous mycobacteria from clinical specimens has also increased, although the incidence of tuberculosis has been greatly reduced. Thanks to a policy granting medical insurance for the performing of liquid culture of tuberculosis bacteria in 2009 in Korea, there was an increase in the number of laboratories performing liquid culture. Liquid culture detects nontuberculous mycobacteria more often than does solid culture. Reports showed that nontuberculous mycobacteria was detected in about 12% of smear-positive tuberculosis cases as measured by liquid culture, with nontuberculous mycobacteria separated from the sputum accounting for about 10˜20% of pulmonary disease cases in Japan, Hong Kong, and Korea, and for about 40˜50% of pulmonary disease in the USA, Canada, and West Europe.

Resembling tuberculosis, which progresses slowly, the pulmonary diseases caused by nontuberculous mycobacteria are apt to be diagnosed wrongly. However, since drugs effective for the inhibition of Mycobacterium tuberculosis are different from those inhibitory of nontuberculous mycobacteria, there is a demand for rapid and accurate separation between tubercle bacilli and nontuberculous mycobacteria so that suitable drugs can be selected.

The conventional reagents in which the primer specific for the genus Mycobacterium is used as the detecting one for nontuberculous mycobacteria are problematic in terms of the accuracy. For example, when only a certain concentration of Mycobacterium tuberculosis is present, the reagents do not respond to primers specific for detecting Mycobacterium tuberculosis, but only to primers for nontuberculous mycobacteria, so Mycobacterium tuberculosis is wrongly identified as nontuberculous mycobacteria. On the other hand, when nontuberculous mycobacteria coexist with Mycobacterium tuberculosis, only nontuberculous mycobacteria are likely to be detected. Therefore, there is a need for a primer set capable of recognizing a nucleotide sequence that is absent from Mycobacterium tuberculosis, but intrinsic to nontuberculous mycobacteria, and for a method for separately detecting Mycobacterium tuberculosis and nontuberculous mycobacteria with rapidity and accuracy using the same.

### DISCLOSURE

### Technical Problem

It is an object of the present invention to provide a primer set specific for the IS6110 or 16S rRNA gene exclusive to Mycobacterium tuberculosis, and a primer set specific for the 16S rRNA gene of nontuberculous mycobacterium, both of which are applicable to the accurate detection and diagnosis of Mycobacterium tuberculosis and nontuberculous mycobacterium, separately.

It is another object of the present invention to provide a kit for the detection of Mycobacterium tuberculosis and nontuberculous mycobacterium, comprising a primer set specific for the IS6110 or 16S rRNA gene exclusive to Mycobacterium tuberculosis, and a primer set specific for the 16S rRNA gene of nontuberculous mycobacterium, both of which are applicable to the accurate detection and diagnosis of Mycobacterium tuberculosis and nontuberculous mycobacterium, separately It is another object of the present invention to provide a method for the detection and diagnosis of Mycobacterium tuberculosis and nontuberculous mycobacteria with accuracy using a duplex polymerase chain reaction based on the primer set.

### Technical Solution

In accordance with an aspect thereof, the present invention provides a primer set specific for the 16S rRNA gene of nontuberculous mycobacteria, comprising a forward primer having the nucleotide sequence of SEQ ID NO: 3; at least one reverse primer selected from the group consisting of nucleotide sequences of SEQ ID NOS: 4 to 6; and a reverse primer having the nucleotide sequence of SEQ ID NO: 7.

In accordance with another aspect thereof, the present invention provides a primer set specific for the IS6110 gene of Mycobacterium tuberculosis, comprising a forward primer having the nucleotide sequence of SEQ ID NO: 10 and a reverse primer having the nucleotide sequence of SEQ ID NO: 11.

In accordance with a further aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

In accordance with still another aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 10 and a reverse primer having the nucleotide sequence of SEQ ID NO: 11; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

In accordance with a still further aspect thereof, the present invention provides a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; and analyzing products of the duplex PCR.

In accordance with yet another aspect thereof, the present invention provides a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 10 and a reverse primer having the nucleotide sequence of SEQ ID NO: 11; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; and analyzing products of the duplex PCR.

In accordance with a yet further aspect thereof, the present invention provides a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 12, and a reverse primer having the nucleotide sequence of SEQ ID NO: 13.

In accordance with yet another aspect thereof, the present invention provides a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 12, and a reverse primer having the nucleotide sequence of SEQ ID NO: 13; and analyzing products of the duplex PCR.

### Advantageous Effects

As described above, primer sets for the detection of Mycobacterium tuberculosis and nontuberculous mycobacteria, capable of detecting specific nucleotide sequences of Mycobacterium tuberculosis and nontuberculous mycobacteria, detection kits, and duplex PCR-based methods for detecting Mycobacterium tuberculosis and nontuberculous mycobacteria using the primer sets, are provided by the present invention. Because the primers are exclusive to Mycobacterium tuberculosis and nontuberculous mycobacteria, the method of the present invention, which can employ an inexpensive typical PCR cycler, can be a clinical diagnosis means useful for the detection of both Mycobacterium tuberculosis and nontuberculous mycobacteria at the same time, with higher efficiency.

### DESCRIPTION OF DRAWINGS

FIG. 1 is a photograph showing DNA fragments run by electrophoresis after duplex PCR using the primer sets of Example 1.

FIG. 2 is a photograph showing DNA fragments run by electrophoresis after duplex PCR using the primer sets of Example 2.

FIG. 3 is a photograph showing DNA fragments run by electrophoresis after duplex PCR using the primer sets of Example 3.

### BEST MODE

In accordance with an aspect thereof, the present invention addresses a primer set specific for the 16S rRNA gene of nontuberculous mycobacteria, comprising a forward primer having the nucleotide sequence of SEQ ID NO: 3; at least one reverse primer selected from the group consisting of nucleotide sequences of SEQ ID NOS: 4 to 6; and a reverse primer having the nucleotide sequence of SEQ ID NO: 7

All of the nucleotide sequences of SEQ ID NOS: 4, 5 and 6 (NTM-1) are reverse primers specific for the 16S rRNA. The nucleotide sequence of SEQ ID NO: 7 (NTM-2) is also a reverse primer specific for the 16S rRNA gene of nontuberculous mycobacteria. All of the reverse primers for the 16S rRNA gene of nontuberculous mycobacteria are designed to detect all of various target nontuberculous mycobacterium species. The reverse primer of SEQ ID NO: 7 (5′-catcccacaccgctaccw-3′) refers to a primer set comprising 5′-catcccacaccgctacct-3′ (SEQ ID NO: 8) and 5′-catcccacaccgctacca-3′ (SEQ ID NO: 9). For example, the nucleotide sequence of SEQ ID NO: 7 may be a primer set in which 5′-catcccacaccgctacct-3′ and 5′-catcccacaccgctacca-3′ are mixed at a ratio of approximately 1:1.

In accordance with another aspect thereof, the present invention addresses a primer set specific for the IS6110 gene of Mycobacterium tuberculosis, comprising a forward primer having the nucleotide sequence of SEQ ID NO: 10 and a reverse primer having the nucleotide sequence of SEQ ID NO: 11. The reverse primer for the 16S rRNA gene of Mycobacterium tuberculosis is designed to detect various mycobacterium species (Mycobacterium tuberculosis complex, MTC).

In accordance with a further aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

The kit for the detection of Mycobacterium tuberculosis and nontuberculous mycobacteria comprises a primer set, specific for the IS6110 of Mycobacterium tuberculosis, which consists of a forward primer and a reverse primer having the nucleotide sequences of SEQ ID NOS: 1 and 2, respectively. The primer set specific for the IS6100 gene is designed to detect various mycobacterium species (Mycobacterium tuberculosis complex, MTC).

The kit for the detection of Mycobacterium tuberculosis and nontuberculous mycobacteria comprises a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6 (NTM-1), and a reverse primer having the nucleotide sequence of SEQ ID NO: 7 (NTM-2). The reverse primers for the 16S rRNA gene of nontuberculous mycobacteria are designed to detect various nontuberculous mycobacterium species.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in size (bp).

In one embodiment, the kit for the detection of Mycobacterium tuberculosis and nontuberculous mycobacteria may comprise the reverse primers having the nucleotide sequences of SEQ ID NOS: 4 and 7 at a ratio of 1:1, the latter being composed of SEQ ID NOS: 8 and 9 at a ratio of 1:1. Accordingly, in the kit, the primer of SEQ ID NO: 4, 5′-catcccacaccgctacct-3′ (SEQ ID NO: 8), and 5′-catcccacaccgctacca-3′ (SEQ ID NO: 9) may be mixed at a ratio of 2:1:1.

In another embodiment of the present invention, the kit for the detection of Mycobacterium tuberculosis and nontuberculous mycobacteria may comprise the reverse primers having the nucleotide sequences of SEQ ID NOS: 5 and 7 at a ratio of 1:1, the latter being composed of SEQ ID NOS: 8 and 9 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 6 and 7 at a ratio of 1:1, the latter being composed of SEQ ID NOS: 8 and 9 at a ratio of 1:1.

In accordance with still another aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 10 and a reverse primer having the nucleotide sequence of SEQ ID NO: 11; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in size (bp).

In one embodiment, the kit for the detection of Mycobacterium tuberculosis and nontuberculous mycobacteria may comprise the reverse primers having the nucleotide sequences of SEQ ID NOS: 4 and 7 at a ratio of 1:1, the latter being composed of SEQ ID NOS: 8 and 9 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 5 and 7 at a ratio of 1:1, the latter being composed of SEQ ID NOS: 8 and 9 at a ratio of 1:1.

In another embodiment of the present invention, the kit for the detection of Mycobacterium tuberculosis and nontuberculous mycobacteria may comprise the reverse primers having the nucleotide sequences of SEQ ID NOS: 6 and 7 at a ratio of 1:1, the latter being composed of SEQ ID NOS: 8 and 9 at a ratio of 1:1.

In accordance with a still further aspect thereof, the present invention addresses a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; and analyzing products of the duplex PCR.

In accordance with yet another aspect thereof, the present invention addresses a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 10 and a reverse primer having the nucleotide sequence of SEQ ID NO: 11; and a primer set specific for the 16S rRNA gene of nontuberculous mycobacteria, composed of a forward primer having the nucleotide sequence of SEQ ID NO: 3, at least one reverse primer selected from the group consisting of nucleotide sequences of SEQ ID NOS: 4 to 6, and a reverse primer having the nucleotide sequence of SEQ ID NO: 7; and analyzing products of the duplex real-time PCR.

In accordance with a yet further aspect thereof, the present invention addresses a kit for the detection of Mycobacterium tuberculosis and nontuberculous mycobacteria, comprising: a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 12, and a reverse primer having the nucleotide sequence of SEQ ID NO: 13.

The kit for the detection of Mycobacterium tuberculosis and nontuberculous mycobacteria comprises a primer set specific for the IS6110 gene of Mycobacterium tuberculosis, composed of a forward primer having the nucleotide sequence of SEQ ID NO: 1, and a reverse primer having the nucleotide sequence of SEQ ID NO: 2. The primer set for the IS6110 gene of Mycobacterium tuberculosis is designed to detect various Mycobacterium species (Mycobacterium tuberculosis complex, MTC).

The kit for the detection of Mycobacterium tuberculosis and nontuberculous mycobacteria comprises a primer set specific for the 16S rRNA gene of nontuberculous mycobacteria, composed of a forward primer having the nucleotide sequence of SEQ ID NO: 12, and a reverse primer having the nucleotide sequence of SEQ ID NO: 13. The forward primer for the 16S rRNA gene of nontuberculous mycobacteria is designed to detect various nontuberculous mycobacterium species.

The detection kit may further comprise a reagent necessary for amplifying DNA by PCR. This reagent may include DNA polymerase, dNTPs, PCR buffer, etc. For use in the kit for the detection of Mycobacterium tuberculosis and nontuberculous mycobacteria, primers are capable of detecting genes characteristic of Mycobacterium tuberculosis and nontuberculous mycobacteria and may be designed to allow for the amplification of PCR products which are greatly different in size (bp).

In the kit for the detection of Mycobacterium tuberculosis and nontuberculous mycobacteria according to an embodiment of the present invention, the forward primer having the nucleotide sequence of SEQ ID NO: 12 is a set of 5′-catgtcttgtgggggaaagctt-3′ (SEQ ID NO: 14), 5′-catgttttgtgggggaaagctt-3′ (SEQ ID NO: 15), 5′-catgtcttctgggggaaagctt-3′ (SEQ ID NO: 16), 5′-catgtcttgtggtggaaagctt-3′ (SEQ ID NO: 17), 5′-catgtcttgtggggcaaagctt-3′ (SEQ ID NO: 18), 5′-catgttttctgggggaaagctt-3′ (SEQ ID NO: 19), 5′-catgtcttctggtggaaagctt-3′ (SEQ ID NO: 20), 5′-catgtcttgtggtgcaaagctt-3′ (SEQ ID NO: 21), 5′-catgttttgtggggcaaagctt-3′ (SEQ ID NO: 22), 5′-catgtcttctggggcaaagctt-3′ (SEQ ID NO: 23), 5′-catgttttgtggtggaaagctt-3′ (SEQ ID NO: 24), 5′-catgttttctggtggaaagctt-3′ (SEQ ID NO: 25), 5′-catgttttctggggcaaagctt-3′ (SEQ ID NO: 26), 5′-catgttttgtggtgcaaagctt-3′ (SEQ ID NO: 27), 5′-catgtcttctggtgcaaagctt-3′ (SEQ ID NO: 28), and 5′-catgttttctggtgcaaagctt-3′ (SEQ ID NO: 29) at a ratio of approximately 1:1:1:1:1:1:1:1:1:1:1:1:1:1:1:1.

In accordance with yet another aspect thereof, the present invention addresses a method for detecting Mycobacterium tuberculosis and nontuberculous mycobacteria, comprising: isolating DNA from a test subject; amplifying the DNA by duplex PCR using a primer set specific for the IS6110 gene of Mycobacterium tuberculosis, composed of a forward primer having the nucleotide sequence of SEQ ID NO: 1 and a reverse primer having the nucleotide sequence of SEQ ID NO: 2; and a primer set specific for the 16S rRNA gene of nontuberculous mycobacteria, composed of a forward primer having the nucleotide sequence of SEQ ID NO: 12, and a reverse primer having the nucleotide sequence of SEQ ID NO: 13; and analyzing products of the duplex PCR.

### MODE FOR INVENTION

A better understanding of the present invention may be obtained through the following examples which are set forth to illustrate, but are not to be construed as limiting the present invention.

### REFERENCE EXAMPLE

### Search for Nucleotide Sequence Characteristic of Mycobacterium tuberculosis and Nontuberculous Mycobacteria

1. Target and Gene Loci

Used in searching for nucleotide sequences characteristic of Mycobacterium tuberculosis and nontuberculous mycobacteria were data of 16S ribosomal RNA genes of the following mycobacteria:

M. abscessus (AJ419970.1, AJ416940.1, AJ536038), M. acapulcensis (AF480575.1), M. africanum (AF480605.1), M. agri (AJ429045.1), M. aichiense (X55598.1), M. alvei (NR_{—}024859.1), M. asiaticum (X55604.1), M. aurum (FJ172298.1), M. austroafricanum (GU121552.1), M. avium (NR_{—}025584.1, AJ536037.1, EF521892.1), M. bohemicum (NR_{—}026054.1), M. botniense (NR_{—}028878.1), M. bovis (GU142937.1), M. branderi (AF480574.1), M. brumae (NR_{—}025233.1), M. celatum (L08169.1), M.chelonae (AM884324.1, AJ419969.1), M. chitae (NR_{—}029220.1), M. chlorophenolicum (NR_{—}026173.1), M. chubuense (X55596.1), M. confluentis (AJ634379.1), M. conspicuum (X029298.1), M. cookii (X53896.1), M. diernhoferi (AF480599.1), M. doricum (NR_{—}025099.1), M. duvalii (NR_{—}026073.1), M. engbaekii (AF480577.1), M. fallax (AF480600.1), M. farcinogenes (X55592.1), M. flavescens (AY734993.1), M. fortuitum (AY457066.1, AF480580.1, GU142933.1), M. gadium (NR_{—}026087.1), M. gastri (GU142918.1), M. genavense (NR_{—}029223.1), M. gilvum (AB491971.1), M. goodii (AY457079.1), M. gordonae (GU142923.1), M. haemophilum (V06638.1), M. hassiacum (NR_{—}026011.1), M. heidelbergense (NR_{—}025268.1), M. hiberniae (NR_{—}026092.1), M. hodleri (NR_{—}026286.1), M. immunogen (AJ011771.1), M. interjectum (X70961.1), M. intermedium (X67847.1), M. intracellulare (AY652958.1, AJ536036.1, X52927.1, M61684.1), M. kansasii (M29575.1, X15916.1), M. lenti flavum (AF480583.1), M. mageritense (AY457076.1), M. malmoense (GQ153278.1), M. marinum (AF456238.1, AY513243.1), M. microti (NR_{—}025234.1), M. monacense (GU142931.1), M. moriokaense (AY859686.1), M. mucogenicum (AF480585.1), M. neoaurum (FJ172306.1), M. nonchromogenicum (DQ058406.1), M. obuense (X55597.1), M. paraffinicum (GQ153282.1), M. parafortuitum (NR_{—}026285.1), M. peregrinum (AY457069.1), M. phlei (AF480603.1), M. porcinum (AY457077.1), M. poriferae (NR_{—}025235.1), M. pulveris (NR_{—}025528.1), M. rhodesiae (NR_{—}025529.1), M. scrofulaceum (GQ153271.1), M. senuense (DQ536409.1), M. septicum (AY457070.1), M. shimoidei (X82459.1), M. simiae (GQ153280.1), M. smegmatis (NR_{—}025311.1), M. sphagni (X55590.1), M. szulgai (X52926.1), M. terrae (NR_{—}029168.1), M. thermoresistibile (GU142928.1), M. tilburgii (AJ580826.1), M. triplex (GQ153279.1), M. triviale (DQ058405.1), M. tuberculosis (GU142936.1, GU142935.1, AY53603.1, X55588.1, X52917.1), M. tusciae (NR_{—}024903.1), M. ulcerans (Z13990.1), M. vaccae (X55601.1), M. wolinskyi (AY457083.1), and M. xenopi (X52929.1). The data of the 16S ribosomal RNA genes were obtained from the database of the NCBI (National center for Biotechnology Information).

Analysis of the data of 16S rRNA gene sequences of the mycobacterium species with the aid of Sequencher 4.9 resulted in characteristic nucleotide sequences, that is, nucleotides characteristic of Mycobacterium tuberculosis complex, and nucleotides absent from Mycobacterium tuberculosis complex, but intrinsic to nontuberculous mycobacteria.

### Example 1

### Separation and Detection of Mycobacterium tuberculosis Complex and Nontuberculous Mycobacteria 1

1. Detection Target and Primer Design

Target genes to be detected were the IS6110 gene for Mycobacterium tuberculosis complex, and the 16S rRNA gene for nontuberculous mycobacteria.

A universal primer was used as a forward primer to amplify the 16S rRNA gene of mycobacteria. NTM-1 and NTM-2, which are characteristic of nontuberculous mycobacteria, were used as reverse primers. These primers useful in the detection of target genes were designed using the Primer3 program.

(1) Mycobacterium Tuberculosis Complex (MTC)

1) target gene: IS6110

2) primers

a. forward primer:
(SEQ ID NO: 1)
5′-cgaactcaaggagcacatca-3′
b. reverse primer:
(SEQ ID NO: 2)
5′-gtcgaggaccatggaggtg-3′

3) PCR product size: 385 bp

(2) Nontuberculous Mycobacteria (NTM)

1) target gene: 16S rRNA

2) primers

a. forward primer:
(SEQ ID NO: 3)
5′-gtggcgaacgggtgagtaa-3′
b. reverse primer
NTM-1:
(SEQ ID NO: 4)
5′-cccacaccgcaaaagctt-3′,
(SEQ ID NO: 5)
5′-cccacaccgcaaaagct-3′,
or
(SEQ ID NO: 6)
5′-tcccacaccgcaaaagct-3′
NTM-2:
(SEQ ID NO: 7)
5′-catcccacaccgctaccw-3′

3) PCR product size: 128 to 135 by

### Example 1-1

### Duplex PCR Using the Nucleotide Sequence of SEQ ID NO: 4 as a Reverse Primer NTM-1 for the Detection of NTM

(1) Isolation of DNA

Mycobacterium tuberculosis ATCC 25177 and Mycobacterium abscessus ATCC 19977 were grown in an exclusive MGIT mycobacteria growth medium using the automated mycobacterial growth system BACTEC MGIT 960 (Becton, Dickinson and Company, Maryland, USA). Of the MGIT broth in which mycobacteria had been cultured, 500 pL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR. As a combined species of MTC and NTM, a mixture of Mycobacterium tuberculosis ATCC 25177 and Mycobacterium abscessus ATCC 19977 was used.

(2) Duplex PCR

Using GeneAmp PCR system 9700 (Applied Biosystems, Foster City, Calif., USA), duplex PCR started with initial denaturation at 94° C. for 2 min and was run with 40 cycles of denaturation at 94° C. for 30 sec, annealing at 56° C. for 30 sec, and extension at 68° C. for 2 min, followed by final extension at 68° C. for 3 min. The composition of the duplex PCR reagent is summarized in Table 1, below. In the primer mix, a forward primer and a reverse primer were contained in the same amounts (10 pmoles/μL). Accordingly, 1.5 μL of the primer mix for MTC contained the forward primer and the reverse primer in an amount of 15 pmoles, each. Since a total volume of PCR mixture was 25 μL, it contained the primers at a concentration of 0.6 μM (15 pmoles/25 μL). For NTM, each of the forward primer and the reverse primer was in an amount of 10 pmoles/1 μL, with a final concentration of 0.4 μM (10 pmoles/25 μL). In this context, each of the NTM-1 reverse primer (SEQ ID NO: 4) and the NTM-2 reverse primer (SEQ ID NO: 7) were used in an amount of 10 pmoles while the NTM-2 reverse primer was comprised of 5 pmoles of 5′-catcccacaccgctacct-3′ (SEQ ID NO: 8) and 5 pmoles of 5′-catcccacaccgctacca-3′ (SEQ ID NO: 9).

TABLE 1

| Ingredient | | Vol. (μL) | Conc. |
|---|---|---|---|
| 10X PCR buffer | | 2.5 | 1X |
| dNTPs mix | | 0.5 | 200 uM |
| (10 mM each, Roche, Mannheim, Germany) | | | |
| Primer Mix (10 pmoles/μL) | MTC | 1.5 | 0.6 uM |
| Primer Mix (10 pmoles/μL) | NTM | 1.0 | 0.4 uM |

| | | |
|---|---|---|
| Taq DNA polymerase | 0.2 | l unit |
| (5 units/μL, Roche, Mannheim, Germany) | | |
| Nuclease free water | 14.3 | — |
| Sample DNA template | 5 | — |
| Total | 25 | — |

### Example 1-2

### Duplex PCR Using the Nucleotide Sequence of SEQ ID NO: 5 as the Reverse Primer NTM-1 for the Detection of NTM

Duplex PCR was carried out in the same manner as in Example 1-1, with the exception that the nucleotide sequence of SEQ ID NO: 5 was used as the reverse primer NTM-1.

### Example 1-3

### Duplex PCR Using the Nucleotide Sequence of SEQ ID NO: 6 as the Reverse Primer NTM-1 for the Detection of NTM

Duplex PCR was carried out in the same manner as in Example 1-1, with the exception that the nucleotide sequence of SEQ ID NO: 6 was used as the reverse primer NTM-1.

2. Result of Duplex PCR

A mixture of 5 pL of the duplex PCR product and 1 pL of a gel loading buffer was loaded to 2% agarose gel containing 1 pg/mL ethidium bromide (EtBr), and run at 157 volt for 25 min by electrophoresis. An image of the electrophoresis was taken using an image analyzer (Bio-Rad, USA) equipped with a UV transilluminator. A size marker was GeneRuler 100 bp DNA Ladder.

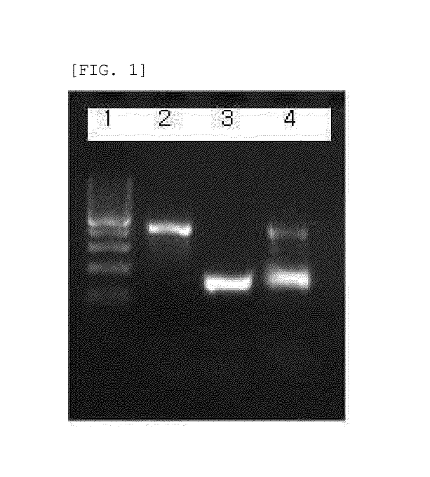

FIG. 1 is a photograph showing DNA fragments run by electrophoresis after duplex PCR using the primer sets of Example 1. The size marker and the DNAs from MTC, NTM, and a combination of MTC+NTM were run in lanes 1, 2, 3, and 4, respectively.

The duplex PCR, as shown in FIG. 1, amplified characteristic target genes which were visualized as a single band at about 385 bp in lane 2, a single band at about 128 bp in lane 3, and a dual band at about 385 bp and about 128 bp in lane 4.

Therefore, the duplex PCR using the primers specific for nucleotide sequences of MTC and NTM was found to guarantee the detection of MTC and NTM from the standard species, simultaneously, with high reliability.

### Example 2

### Separation and Detection of Mycobacterium tuberculosis Complex and Nontuberculous Mycobacteria 2

1. Detection Target and Primer Design

Target genes to be detected were the 16S rRNA gene for both Mycobacterium tuberculosis complex, and nontuberculous mycobacteria. The primers useful in the detection of the target genes were designed using the Primer3 program.

(1) Mycobacterium tuberculosis (MTC)

1) target gene: 16S rRNA

2) primers

a. forward primer:
(SEQ ID NO: 10)
5′-gtcttgtggtggaaagcgc-3′
b. reverse primer:
(SEQ ID NO: 11)
5′-agagaacccggaccttcgt-3′

3) PCR product size: 272 by

(2) Nontuberculous Mycobacteria (NTM)

1) target gene: 16S rRNA

2) primers

a. forward primer:
(SEQ ID NO: 3)
5′-gtggcgaacgggtgagtaa-3′
b. reverse primer
NTM-1:
(SEQ ID NO: 4)
5′-cccacaccgcaaaagctt-3′,
(SEQ ID NO: 5)
5′-cccacaccgcaaaagct-3′,
or
(SEQ ID NO: 6)
5′-tcccacaccgcaaaagct-3′
NTM-2:
(SEQ ID NO: 7)
5′-catcccacaccgctaccw-3′

3) PCR product size: 128 to 135 by

### Example 2-1

### Duplex PCR Using the Nucleotide Sequence of SEQ ID NO: 4 as a Reverse Primer NTM-1 for the Detection of NTM

(1) Isolation of DNA

Mycobacterium tuberculosis ATCC 25177 and Mycobacterium abscessus ATCC 19977 were grown in an exclusive MGIT mycobacteria growth medium using the automated mycobacterial growth system BACTEC MGIT 960 (Becton, Dickinson and Company, Maryland, USA). Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR. As a combined species of MTC and NTM, a mixture of Mycobacterium tuberculosis ATCC 25177 and Mycobacterium abscessus ATCC 19977 was used.

(2) Duplex PCR

Using GeneAmp PCR system 9700 (Applied Biosystems, Foster City, Calif., USA), duplex PCR started with initial denaturation at 94° C. for 2 min and was run with 40 cycles of denaturation at 94° C. for 30 sec, annealing at 56° C. for 30 sec, and extension at 68° C. for 2 min, followed by final extension at 68° C. for 3 min. The composition of the duplex PCR reagent is summarized in Table 2, below. In the primer mix, a forward primer and a reverse primer were contained in the same amounts (10 pmoles/μL). Accordingly, 1.5 μL of the primer mix for MTC contained the forward primer and the reverse primer in an amount of 15 pmoles, each. Since a total volume of PCR mixture was 25 μL, it contained the primers at a concentration of 0.6 μM (15 pmoles/25 μL). For NTM, each of the forward primer and the reverse primer was in an amount of 10 pmoles/1 μL, with a final concentration of 0.4 μM (10 pmoles/25 μL). In this context, each of the NTM-1 reverse primer (SEQ ID NO: 4) and the NTM-2 reverse primer (SEQ ID NO: 7) were used in an amount of 10 pmoles while the NTM-2 reverse primer was comprised of 5 pmoles of 5′-catcccacaccgctacct-3′ (SEQ ID NO: 8) and 5 pmoles of 5′-catcccacaccgctacca-3′ (SEQ ID NO: 9).

TABLE 2

| Ingredient | | Vol. (μL) | Conc. |
|---|---|---|---|
| 10X PCR buffer | | 2.5 | 1X |
| dNTPs mix | | 0.5 | 200 uM |
| (10 mM each, Roche, Mannheim, Germany) | | | |
| Primer Mix (10 pmole/μL) | MTC | 1.5 | 0.6 uM |
| Primer Mix (10 pmole/μL) | NTM | 1.0 | 0.4 uM |

| | | |
|---|---|---|
| Taq DNA polymerase | 0.2 | 1 unit |
| (5 units/μL, Roche, Mannheim, Germany) | | |
| Nuclease free water | 14.3 | — |
| Sample DNA template | 5 | — |
| Total | 25 | — |

### Example 2-2

### Duplex PCR Using the Nucleotide Sequence of SEQ ID NO: 5 as the Reverse Primer NTM-1 for the Detection of NTM

Duplex PCR was carried out in the same manner as in Example 2-1, with the exception that the nucleotide sequence of SEQ ID NO: 5 was used as the reverse primer NTM-1.

### Example 2-3

### Duplex PCR Using the Nucleotide Sequence of SEQ ID NO: 6 as the Reverse Primer NTM-1 for the Detection of NTM

Duplex PCR was carried out in the same manner as in Example 2-1, with the exception that the nucleotide sequence of SEQ ID NO: 6 was used as the reverse primer NTM-1.

2. Result of Duplex PCR

A mixture of 5 μL of the duplex PCR product and 1 μL of a gel loading buffer was loaded to 2% agarose gel containing 1 μg/mL ethidium bromide (EtBr), and run at 157 volt for 25 min by electrophoresis. An image of the electrophoresis was taken using an image analyzer (Bio-Rad, USA) equipped with a UV transilluminator. A size marker was GeneRuler 100 by DNA Ladder.

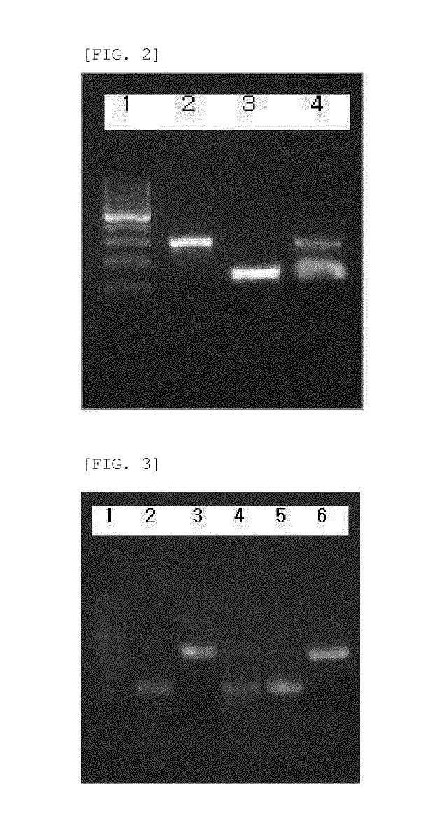

FIG. 2 is a photograph showing DNA fragments run by electrophoresis after duplex PCR using the primer sets of Example 2. The size marker and the DNAs from MTC, NTM, and a combination of MTC+NTM were run in lanes 1, 2, 3, and 4, respectively.

The duplex PCR, as shown in FIG. 2, amplified characteristic target genes which were visualized as a single band at about 272 bp in lane 2, a single band at about 128 bp in lane 3, and a dual band at about 128 bp and about 272 bp in lane 4.

Therefore, the duplex PCR using the primers specific for nucleotide sequences of MTC and NTM was found to guarantee the detection of MTC and NTM from the standard species, simultaneously, with high reliability.

### Example 3

### Separation and Detection of Mycobacterium tuberculosis Complex and Nontuberculous Mycobacteria 3

1. Detection Target and Primer Design

Target genes to be detected were the IS6110 gene for Mycobacterium tuberculosis complex, and the 16S rRNA gene for nontuberculous mycobacteria. For use in the detection of nontuberculous mycobacteria, a forward primer was specific for the target gene of nontuberculous mycobacteria while a universal primer was used as a reverse primer. These primers useful in the detection of target genes were designed using the Primer3 program.

(1) Mycobacterium Tuberculosis Complex (MTC)

1) target gene: IS6110

2) primers

a. forward primer:
(SEQ ID NO: 1)
5′-cgaactcaaggagcacatca-3′
b. reverse primer:
(SEQ ID NO: 2)
5′-gtcgaggaccatggaggtg-3′

3) PCR product size: 385 by

(2) Nontuberculous Mycobacteria (NTM)

1) target gene: 16S rRNA

2) primers

a. forward primer:
(SEQID NO: 12)
5′-catgtyttstggkgsaaagctt-3′
b. reverse primer:
(SEQ ID NO: 13)
5′-cgtaggagtctgggccgta-3′

3) PCR product size: 152 by

2. Duplex PCR

(1) Isolation of DNA

The four standard species Mycobacterium tuberculosis ATCC 25177, Mycobacterium microti ATCC 19422, Mycobacterium abscessus ATCC 19977 and Mycobacterium haemophilum ATCC 29548, and Mycobacterium species and nontuberculous mycobacterium species, identified in clinical subjects, were employed. These bacteria were grown in an exclusive MGIT mycobacteria growth medium using the automated mycobacterial growth system BACTEC MGIT 960 (Becton, Dickinson and Company, Maryland, USA). Of the MGIT broth in which mycobacteria had been cultured, 500 μL was transferred into a 1.5 mL tube, and centrifuged at 14,000 rpm for 5 min. The supernatant was removed, and the pellet was dissolved in 300 μL of sterile distilled water and heated for 10 min in a boiling water bath. Following centrifugation at 14,000 rpm for 5 min, the supernatant was used as a template in PCR.

(2) Duplex PCR

Using GeneAmp PCR system 9700 (Applied Biosystems, Foster City, Calif., USA), duplex PCR started with initial denaturation at 94° C. for 2 min and was run with 40 cycles of denaturation at 94° C. for 30 sec, annealing at 52° C. for 30 sec, and extension at 65° C. for 2 min, followed by final extension at 65° C. for 3 min. The composition of the duplex PCR reagent is summarized in Table 3, below. In the primer mix, a forward primer and a reverse primer were contained in the same amounts (10 pmoles/μL). Accordingly, 1.25 μL of the primer mix for MTC contained the forward primer and the reverse primer in an amount of 12.5 pmoles, each. Since a total volume of PCR mixture was 25 μL, it contained the primers at a concentration of 0.5 μM (12.5 pmoles/25 μL). For NTM, each of the forward primer and the reverse primer was in an amount of 15 pmoles/1.5 μL, with a final concentration of 0.6 μM (15 pmoles/25 μL). In this context, the NTM forward primer was comprised of 5′-catgtcttgtgggggaaagctt-3′ (SEQ ID NO: 14), 5′-catgttttgtgggggaaagctt-3′ (SEQ ID NO: 15), 5′-catgtcttctgggggaaagctt-3′ (SEQ ID NO: 16), 5′-catgtcttgtggtggaaagctt-3′ (SEQ ID NO: 17), 5′-catgtcttgtggggcaaagctt-3′ (SEQ ID NO: 18), 5′-catgttttctgggggaaagctt-3′ (SEQ ID NO: 19), 5′-catgtcttctggtggaaagctt-3′ (SEQ ID NO: 20), 5′-catgtcttgtggtgcaaagctt-3′ (SEQ ID NO: 21), 5′-catgttttgtggggcaaagctt-3′ (SEQ ID NO: 22), 5′-catgtcttctggggcaaagctt-3′ (SEQ ID NO: 23), 5′-catgttttgtggtggaaagctt-3′ (SEQ ID NO: 24), 5′-catgttttctggtggaaagctt-3′ (SEQ ID NO: 25), 5′-catgttttctggggcaaagctt-3′ (SEQ ID NO: 26), 5′-catgttttgtggtgcaaagctt-3′ (SEQ ID NO: 27), 5′-catgtcttctggtgcaaagctt-3′ (SEQ ID NO: 28) and 5′-catgttttctggtgcaaagctt-3′ (SEQ ID NO: 29) at a ratio of approximately 1:1:1:1:1:1:1:1:1:1:1:1:1:1:1:1.

TABLE 3

| Ingredient | | Vol. (μL) | Conc. |
|---|---|---|---|
| 10X PCR buffer | | 2.5 | 1X |
| dNTPs mix | | 0.5 | 200 uM |
| (10 mM, each, Roche, Mannheim, Germany) | | | |
| Primer Mix (10 pmoles/μL) | MTC | 1.25 | 0.5 uM |
| Primer Mix (10 pmoles/μL) | NTM | 1.5 | 0.6 uM |

| | | |
|---|---|---|
| Taq DNA polymerase | 0.2 | l unit |
| (5units/μL, Roche, Mannheim, Germany) | | |
| Nuclease free water | 14.05 | — |
| Sample DNA template | 5 | — |
| Total | 25 | — |

3. Result of Duplex PCR

A mixture of 5 μL of the duplex PCR product and 1 μL of a gel loading buffer was loaded to 2% agarose gel containing 1 μg/mL ethidium bromide (EtBr), and run at 157 volt for 25 min by electrophoresis. An image of the electrophoresis was taken using an image analyzer (Bio-Rad, USA) equipped with a UV transilluminator. A size marker was GeneRuler 100 by DNA Ladder.

FIG. 3 is a photograph showing DNA fragments run by electrophoresis after duplex PCR using the primer sets of Example 3. The size marker and the DNAs from NTM (M. haemophilum), MTC (M. microti), clinical subject (MTC+NTM), NTM (M. abscessus), and MTC (M. tuberculosis) were run in lanes 1 to 6, respectively.

The duplex PCR, as shown in FIG. 3, amplified characteristic target genes which were visualized as a single band at about 385 bp in lanes 3 and 6, a single band at about 152 bp in lanes 2 and 6, and a dual band at about 152 bp and about 385 by in lane 4.

Therefore, the duplex PCR using the primers specific for nucleotide sequences of MTC and NTM was found to guarantee the detection of MTC and NTM from the standard species, simultaneously, with high reliability.

In combination with detection kits comprising forward and reverse primers designed on the basis of nucleotide sequences which are characteristic of MTC, or which are absent from MTC but intrinsic to NTM, as demonstrated in the Examples, duplex PCR can be used to detect MTC and NTM with high reliability. Therefore, the present invention provides a means for detecting MTC and NTM effectively.

Being capable of detecting nucleotide sequences of MTC and NTM, the primer sets of the present invention are highly sensitive to and selective for MTC and NTM when applied to detection, as described above. In addition, the duplex PCR using the primers according to the present invention is a clinical diagnostics means that promises the very effective, simultaneous detection of both MTC and NTM in a target subject.

### INDUSTRIAL APPLICABILITY

Useful in detecting genes characteristic of MTC and NTM, as described hitherto, the primer sets, detection kits, and detection methods according to the present invention can be applied to the clinical diagnosis of diseases caused by MTC and NTM, and therefore find applications in the medical fields including hospitals, research institutes, etc.

### Sequence List text

The nucleotide sequence of SEQ ID NO: 1 is a forward primer specific for the IS6110 gene of Mycobacterium tuberculosis complex.

The nucleotide sequence of SEQ ID NO: 2 is a reverse primer specific for the IS6110 gene of Mycobacterium tuberculosis complex.

The nucleotide sequence of SEQ ID NO: 3 is a reverse primer specific for the 16S rRNA gene of nontuberculous mycobacteria.

The nucleotide sequence of SEQ ID NO: 4 is a reverse primer specific for the 16S rRNA gene of nontuberculous mycobacteria (NTM-1).

The nucleotide sequence of SEQ ID NO: 5 is a reverse primer specific for the 16S rRNA gene of nontuberculous mycobacteria

(NTM-1).

The nucleotide sequence of SEQ ID NO: 6 is a reverse primer specific for the 16S rRNA gene of nontuberculous mycobacteria (NTM-1).

The nucleotide sequence of SEQ ID NO: 7 is a reverse primer specific for the 16S rRNA gene of nontuberculous mycobacteria (NTM-2).

The nucleotide sequence of SEQ ID NO: 8 is a reverse primer specific for the 16S rRNA gene of nontuberculous mycobacteria (NTM-2).

The nucleotide sequence of SEQ ID NO: 9 is a reverse primer specific for the 16S rRNA gene of nontuberculous mycobacteria (NTM-2).

The nucleotide sequence of SEQ ID NO: 10 is a forward primer specific for the 16S rRNA gene of Mycobacterium tuberculosis complex.

The nucleotide sequence of SEQ ID NO: 11 is a reverse primer specific for the 16S rRNA gene of Mycobacterium tuberculosis complex.

The nucleotide sequence of SEQ ID NO: 12 is a forward primer specific for the 16S rRNA gene of nontuberculous mycobacteria.

The nucleotide sequence of SEQ ID NO: 13 is a reverse primer specific for the 16S rRNA gene of nontuberculous mycobacteria.

The nucleotide sequence of SEQ ID NO: 14 is a forward primer specific for the 16S rRNA gene of nontuberculous mycobacteria.

The nucleotide sequence of SEQ ID NO: 15 is a forward primer specific for the 16S rRNA gene of nontuberculous mycobacteria.

The nucleotide sequence of SEQ ID NO: 16 is a forward primer specific for the 16S rRNA gene of nontuberculous mycobacteria.

The nucleotide sequence of SEQ ID NO: 17 is a forward primer specific for the 16S rRNA gene of nontuberculous mycobacteria.

The nucleotide sequence of SEQ ID NO: 18 is a forward primer specific for the 16S rRNA gene of nontuberculous mycobacteria.

The nucleotide sequence of SEQ ID NO: 19 is a forward primer specific for the 16S rRNA gene of nontuberculous mycobacteria.

The nucleotide sequence of SEQ ID NO: 20 is a forward primer specific for the 16S rRNA gene of nontuberculous mycobacteria.

The nucleotide sequence of SEQ ID NO: 21 is a forward primer specific for the 16S rRNA gene of nontuberculous mycobacteria.

The nucleotide sequence of SEQ ID NO: 22 is a forward primer specific for the 16S rRNA gene of nontuberculous mycobacteria.

The nucleotide sequence of SEQ ID NO: 23 is a forward primer specific for the 16S rRNA gene of nontuberculous mycobacteria.

The nucleotide sequence of SEQ ID NO: 24 is a forward primer specific for the 16S rRNA gene of nontuberculous mycobacteria.

The nucleotide sequence of SEQ ID NO: 25 is a forward primer specific for the 16S rRNA gene of nontuberculous mycobacteria.

The nucleotide sequence of SEQ ID NO: 26 is a forward primer specific for the 16S rRNA gene of nontuberculous mycobacteria.

The nucleotide sequence of SEQ ID NO: 27 is a forward primer specific for the 16S rRNA gene of nontuberculous mycobacteria.

The nucleotide sequence of SEQ ID NO: 28 is a forward primer specific for the 16S rRNA gene of nontuberculous mycobacteria.

The nucleotide sequence of SEQ ID NO: 29 is a forward primer specific for the 16S rRNA gene of nontuberculous mycobacteria.

1. A primer set specific for a 16S rRNA gene of nontuberculous mycobacteria, comprising:
a forward primer having a nucleotide sequence of SEQ ID NO: 3;
at least one reverse primer selected from the group consisting of nucleotide sequences of SEQ ID NOS: 4 to 6; and
a reverse primer having a nucleotide sequence of SEQ ID NO: 7

2. A primer set specific for an IS6110 gene of Mycobacterium tuberculosis, comprising a forward primer having a nucleotide sequence of SEQ ID NO: 10 and a reverse primer having a nucleotide sequence of SEQ ID NO: 11.

3. A kit for detection of Mycobacterium tuberculosis and nontuberculous mycobacteria, comprising:
a primer set specific for an IS6110 gene of Mycobacterium tuberculosis, composed of a forward primer having a nucleotide sequence of SEQ ID NO: 1 and a reverse primer having a nucleotide sequence of SEQ ID NO: 2; and
a primer set specific for the 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7.

4. A kit for detection of Mycobacterium tuberculosis and nontuberculous mycobacteria, comprising:
a primer set specific for an IS6110 gene of Mycobacterium tuberculosis, composed of a forward primer having a nucleotide sequence of SEQ ID NO: 10 and a reverse primer having a nucleotide sequence of SEQ ID NO: 11; and
a primer set specific for an 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7.

5. A method for detecting Mycobacterium tuberculosis and nontuberculous mycobacteria, comprising:
isolating DNA from a test subject;
amplifying the DNA by duplex PCR using a primer set specific for an IS6110 gene of Mycobacterium tuberculosis, composed of a forward primer having the nucleotide sequence of SEQ ID NO: 1 and a reverse primer having a nucleotide sequence of SEQ ID NO: 2; and a primer set specific for a 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7; and
analyzing products of the duplex PCR.

6. A method for detecting Mycobacterium tuberculosis and nontuberculous mycobacteria, comprising:
isolating DNA from a test subject;
amplifying the DNA by duplex PCR using a primer set specific for an IS6110 gene of Mycobacterium tuberculosis, composed of a forward primer having a nucleotide sequence of SEQ ID NO: 10 and a reverse primer having a nucleotide sequence of SEQ ID NO: 11; and a primer set specific for an 16S rRNA gene of nontuberculous mycobacteria, composed of a forward primer having a nucleotide sequence of SEQ ID NO: 3, at least one reverse primer selected from the group consisting of nucleotide sequences of SEQ ID NOS: 4 to 6, and a reverse primer having a nucleotide sequence of SEQ ID NO: 7; and
analyzing products of the duplex PCR.

7. A kit for detection of Mycobacterium tuberculosis and nontuberculous mycobacteria, comprising:
a primer set specific for an IS6110 gene of Mycobacterium tuberculosis, composed of a forward primer having a nucleotide sequence of SEQ ID NO: 1 and a reverse primer having a nucleotide sequence of SEQ ID NO: 2; and
a primer set specific for a 16S rRNA gene of nontuberculous mycobacteria, composed of a forward primer having a nucleotide sequence of SEQ ID NO: 12, and a reverse primer having a nucleotide sequence of SEQ ID NO: 13.

8. A method for detecting Mycobacterium tuberculosis and nontuberculous mycobacteria, comprising:
isolating DNA from a test subject;
amplifying the DNA by duplex PCR using a primer set specific for an IS6110 gene of Mycobacterium tuberculosis, composed of a forward primer having a nucleotide sequence of SEQ ID NO: 1 and a reverse primer having a nucleotide sequence of SEQ ID NO: 2; and a primer set specific for a 16S rRNA gene of nontuberculous mycobacteria, composed of a forward primer having a nucleotide sequence of SEQ ID NO: 12, and a reverse primer having a nucleotide sequence of SEQ ID NO: 13; and
analyzing products of the duplex PCR.
